(19) United States
(12) Patent Application Publication
(10) Pub. No.: US 2024/0281977 A1
(43) Pub. Date: Aug. 22, 2024

(54) METHOD AND APPARATUS FOR ANALYZING INTRACORONARY IMAGES

(71) Applicant: CAMBRIDGE ENTERPRISE LIMITED, Cambridge (GB)

(72) Inventors: Adam WOOLF, London (GB); Martin BENNETT, London (GB)

(21) Appl. No.: 18/652,327

(22) Filed: May 1, 2024

Related U.S. Application Data
(63) Continuation-in-part of application No. 17/606,996, filed on Oct. 27, 2021, now Pat. No. 11,983,875.

(30) Foreign Application Priority Data
May 1, 2019 (GB) 1906103.5

(51) Int. Cl. G06T 7/00; G06T 5/20; G06T 5/40; G06T 5/60; G06T 7/11; G06T 7/13; G06T 7/136; G06V 10/26; G06V 10/32; G06V 10/36; G06V 10/54; G06V 10/75; G06V 10/764; G06V 10/82; G16H 50/20

(52) U.S. Cl. CPC G06T 7/0016; G06T 5/20; G06T 5/40; G06T 5/60; G06T 7/11; G06T 7/13; G06T 7/136; G06V 10/26; G06V 10/32; G06V 10/36; G06V 10/54; G06V 10/758; G06V 10/764; G06V 10/82; G16H 50/20; G06T 2207/10101; G06T 2207/20004; G06T 2207/20032; G06T 2207/20036; G06T 2207/20081; G06T 2207/30048; G06T 2207/30101; G06V 2201/03

(57) ABSTRACT

Disclosed is a method for analyzing a set of images of a coronary artery tissue. The method comprises segmenting the images for the presence of normal artery features and those associated with OCT, correcting artifacts, and optimizing the images. The method further comprises segmenting the diseased tissue into distinct tissue types, and measuring features of interests of the segmented tissue types. The method further comprises compiling a first set of measurements for each identified feature of interest at a first time, and a second set of measurements at a second time subsequent to the first time. The method further comprises determining changes in the coronary artery tissue, indicative of progression or regression of a diseased state, or prediction of multiple adverse cardiovascular events (MACE) such as cardiac death or myocardial infarction.

### CROSS-REFERENCE TO RELATED APPLICATIONS

This application is a continuation-in-part of U.S. patent application Ser. No. 17/606,996, having a § 371(c) date of Oct. 27, 2021, which itself is a 35 U.S.C. § 371 national stage application of PCT Application No. PCT/GB2020/051051, filed on Apr. 30, 2020, which itself claims priority from Great Britain Patent Application No. 1906103.5, filed on May 1, 2019, the contents of all of which are incorporated herein by reference in their entireties.

### FIELD

The present disclosure relates generally to the field of medical imaging analysis. More specifically, the present disclosure pertains to an automated computer-implemented method for analyzing intracoronary optical coherence tomography (OCT) images to evaluate coronary artery tissue, identify the efficacy of drug or device therapy, and predict patient outcomes based on detected tissue characteristics.

### BACKGROUND

Coronary artery disease (CAD) imaging has the potential to identify high-risk atherosclerotic plaques and is a widely used surrogate efficacy marker for drug and device studies. However, event rates of presumed ‘high-risk’ lesions identified using different imaging modalities are below those needed to change management of individual plaques, and modalities are suboptimal to predict which plaques will cause future adverse coronary events, with positive predictive values of only 20-30%. Intracoronary optical coherence tomography (OCT) produces very high-resolution (10-20 μm) sequences that can provide exquisitely detailed plaque images. Furthermore, a number of OCT parameters are associated with high-risk lesions, including: minimum fibrous cap thickness (FCT), minimum lumen area (MLA), lipid arc, and presence of macrophages, calcific nodules, neovascularization, or cholesterol crystals. Some of these OCT features change with treatment with drugs such as high-dose statins or ezetimibe, agents that reduce patient events with minimal changes in plaque volume, suggesting that they may induce plaque stabilization.

However, OCT pullbacks are rich datasets containing hundreds of images and tens-of-thousands of candidate measurements per artery. Consequently, detailed OCT analysis currently requires time-consuming offline manual frame selection and measurement in specialized core laboratories, and can be limited by inter- and intra-observer variability and the high frequency of artifacts and similarity of artifact to disease. In contrast, a fully automated, time-efficient OCT analysis system that can optimize images and reduce artifacts could improve utilization of this versatile technology. While several fully automated systems have been built and tested for accuracy, it is unclear whether they can identify disease progression/regression in clinical trials and/or higher-risk plaques to guide patient management. Additionally, histopathological validation of many analysis systems is lacking as is external validation against core laboratories using large-scale clinical trial data to provide model performance. Finally, many models have been developed with small or highly selected training datasets, for example, with the exclusion of OCT frames containing stents or with poor image quality or artifacts, which may limit their generalizability and accuracy in real-world clinical practice.

Given these challenges, there is a clear need for an improved method for analyzing intracoronary OCT images that can handle the complexity of OCT datasets efficiently and accurately. Such a method should be able to distinguish diseased tissue from artifacts and also be validated against clinical trial data to ensure its efficacy in real-world applications. The present disclosure addresses this need by providing an automated, robust, and clinically validated method that enhances the analysis of OCT images, supports the evaluation of therapeutic interventions, and guides patient management with greater precision and reliability.

### SUMMARY

The present disclosure advances the field of medical imaging by introducing a refined automated method for analyzing intracoronary optical coherence tomography (OCT) images to assess the coronary artery anatomy and disease. This computer-implemented method utilizes a sequence of neural networks that systematically processes OCT images to discern between healthy and diseased tissue, identify artifacts, and conduct a comprehensive analysis of the coronary artery's condition.

The present method involves the classification of the OCT images for the presence of diseased tissue using the first neural network. The method, then, progresses to the segmentation of the artery, including lumen, side branches, stent/guide catheter, guidewire shadows, and external elastic lamina (EEL). Following this determination, a second neural network further classifies the images to detect the presence of artifacts. The present method implements artifact correction methodology to rectify artifacts through a series of sophisticated image processing techniques. After artifact correction, the method includes segmenting the diseased tissue into distinct tissue types, such as fibrous, lipid-rich, and calcific tissues. This segmentation is executed by employing a third neural network, specifically tailored to recognize and differentiate between these tissue types within the OCT images. Following segmentation, the method includes a step for the identification and measurement of critical features of interest within the segmented tissues, including high-risk plaque features (see above). For sequential analysis studies, the method further comprises the compilation of measurements from two sets of images: one set captured at a first time and a subsequent set captured at a second time. The method utilizes these compiled measurements to determine changes in the coronary artery and tissue over time, offering insights into disease progression or regression. These changes are critical indicators for the efficacy assessment of drug or device therapies used in treating coronary artery disease.

The artifact correction methodology involves converting the OCT images to greyscale, performing lumen masking, applying a polar transform from a central point in the lumen to produce panels, measuring the mean pixel intensity of each panel, carrying out histogram matching to align pixel intensities, integrating the matched panels into a single image, reconstructing the image with a Cartesian transform, and normalizing the images for artifact correction. The artifact correction methodology further incorporates the application of a median filter to the integrated image, which serves to enhance the clarity of the OCT image. The artifact correction methodology also includes the additional step of using adaptive filtering with binary masks derived from thresholding operations, which is applied during the processing of the set of OCT images.

The present method also involves configuring the second neural network with machine learning algorithms capable of identifying artifacts by analyzing patterns indicative of distortions and categorizing these artifacts into correctable and non-correctable types.

The present method further involves configuring the third neural network with a convolutional neural network (CNN) architecture designed to extract and organize features from the OCT images, which assists in the segmentation of the coronary artery tissue into distinct tissue types.

The present method may further include the implementation of spatial filtering, intensity normalization, and edge detection techniques, which aid in facilitating the segmentation of tissue.

The present method also involves the use of a fourth neural network that analyzes the compiled measurements to determine the efficacy of drug or device therapy by employing a predictive model trained on historical data correlating tissue characteristics with patient outcomes.

In the present method, measuring the features of interest of the segmented tissue types involves quantifying the morphological and textural properties of each tissue type to assess plaque composition, including assessment for the presence of high-risk features.

In the present method, the set of images include OCT images that may be pre-processed to normalize lighting conditions and contrast levels before analysis. The method includes aligning and co-registering the first and second subsets of images using anatomical landmarks within the coronary artery tissue.

In the present method, each image of the coronary artery tissue is used to determine the likelihood of a patient having a particular manifestation of coronary artery disease or patient event. The present method uses the identified features of interest, including but not limited to: plaque type, lipid arc, lumen area, and fibrous cap thickness, in the diagnostic process, and focuses on cardiac death and myocardial infarction as patient events, but may also include other events such as admission to hospital and revascularization.

Another application is a method of treatment of a patient comprising diagnosing a patient with one or more cardiac diseases (e.g., stable angina, non-ST elevation myocardial infarction (NSTEMI) or ST elevation myocardial infarction (STEMI)) and treating the patient based on the diagnosis. Similarly, another application is a method of treatment of a patient predicted to have a high likelihood of a particular cardiac event and treating the patient based on the prediction, for example with revascularization or additional medical treatments.

This automated method addresses the challenges of time-intensive and variability-prone manual analysis methods, providing a reliable, efficient, and accurate tool for the analysis of OCT images. The present method significantly contributes to the field by improving the accuracy of diagnostic imaging and the prediction of drug and device efficacy and patient outcomes, thereby influencing therapeutic strategies and advancing patient care in coronary artery disease management.

As will be appreciated by one skilled in the art, the present disclosure may be embodied as a system, method or computer program product. Accordingly, the present disclosure may take the form of an entirely hardware embodiment, an entirely software embodiment, or an embodiment combining software and hardware aspects.

Furthermore, the present disclosure may take the form of a computer program product embodied in a computer-readable medium having computer-readable program code embodied thereon. The computer-readable medium may be a computer-readable signal medium or a computer-readable storage medium. A computer-readable medium may be, for example, but is not limited to, an electronic, magnetic, optical, electromagnetic, infrared, or semiconductor system, apparatus, or device, or any suitable combination of the foregoing.

Computer program code for carrying out operations of the present disclosure may be written in any combination of one or more programming languages, including object-oriented programming languages and conventional procedural programming languages. Code components may be embodied as procedures, methods or the like, and may comprise sub-components that may take the form of instructions or sequences of instructions at any of the levels of abstraction, from the direct machine instructions of a native instruction set to high-level compiled or interpreted language constructs.

Embodiments of the present disclosure also provide a non-transitory data carrier carrying code which, when implemented on a processor, causes the processor to carry out any of the methods described herein.

The present disclosure further provides processor control code to implement the methods described herein, for example on a general-purpose computer system or on a digital signal processor (DSP). The present disclosure also provides a carrier carrying processor control code to, when running, implement any of the methods described herein, in particular on a non-transitory data carrier. The code may be provided on a carrier such as a disk, a microprocessor, CD- or DVD-ROM, programmed memory such as non-volatile memory (e.g. Flash) or read-only memory (firmware), or on a data carrier such as an optical or electrical signal carrier. Code (and/or data) to implement embodiments of the techniques described herein may comprise source, object or executable code in a conventional programming language (interpreted or compiled) such as python, C, or assembly code, code for setting up or controlling an ASIC (Application Specific Integrated Circuit) or FPGA (Field Programmable Gate Array), or code for a hardware description language such as Verilog® or VHDL (Very high speed integrated circuit Hardware Description Language). As the skilled person will appreciate, such code and/or data may be distributed between a plurality of coupled components in communication with one another. The present disclosure may comprise a controller which includes a microprocessor, working memory and program memory coupled to one or more of the components of the system.

It will also be clear to one skilled in the art that all or part of a logical method according to embodiments of the present disclosure may suitably be embodied in a logic apparatus comprising logic elements to perform the steps of the above-described methods, and that such logic elements may comprise components such as logic gates in, for example a programmable logic array or application-specific integrated circuit. Such a logic arrangement may further be embodied in enabling elements for temporarily or permanently establishing logic structures in such an array or circuit using, for example, a virtual hardware descriptor language, which may be stored and transmitted using fixed or transmittable carrier media.

In an embodiment, the present disclosure may be implemented using multiple processors or control circuits. The present disclosure may be adapted to run on, or integrated into, the operating system of an apparatus.

In an embodiment, the present disclosure may be realized in the form of a data carrier having functional data thereon, said functional data comprising functional computer data structures to, when loaded into a computer system or network and operated upon thereby, enable said computer system to perform all the steps of the methods described herein.

### BRIEF DESCRIPTION OF THE DRAWINGS

For a better understanding, and to show how embodiments may be carried into effect, reference will now be made, by way of example only, to the accompanying diagrammatic drawings in which:

FIG. 1 is a flowchart of an automated computer-implemented method for analyzing a set of images of a coronary artery tissue, in accordance with one or more embodiments of the present disclosure;

FIG. 2 illustrates an artery from which a set of images have been captured, in accordance with one or more embodiments of the present disclosure;

FIG. 3 illustrates schematic of a system for capturing and processing an image captured from the artery, in accordance with one or more embodiments of the present disclosure;

FIG. 4 illustrate various stages involved in the automated computer-implemented method for analyzing intracoronary optical coherence tomography (OCT) images for assessing coronary artery disease, in accordance with one or more embodiments of the present disclosure;

FIGS. 5A-5D illustrates an outline of a deep learning model used in the automated method for processing and analyzing intracoronary OCT images, and their segmentation for histopathology (FIG. 5A) and clinical scans (FIG. 5B), plaque type/classification (FIG. 5C) and validation against expert readers for histopathology and clinical scans, or core laboratory analyses for clinical trials (FIG. 5D), in accordance with one or more embodiments of the present disclosure;

FIGS. 6A-6D illustrate results of the segmentation process applied in the automated analysis of intracoronary OCT images, in accordance with one or more embodiments of the present disclosure;

FIGS. 7A-7C illustrate an overview of measurements from the automated method for analyzing intracoronary OCT images in lipid-containing lesions, in accordance with one or more embodiments of the present disclosure;

FIGS. 8A-8B illustrate an overview of measurements from the automated method in measuring features of interest within the coronary artery tissue vs. an expert clinical reader, in accordance with one or more embodiments of the present disclosure;

FIGS. 9A and 9B present graphical representation that illustrates the changes in the minimum thickness of the fibrous cap of individual coronary artery plaques over time with drug treatment, in accordance with one or more embodiments of the present disclosure.

FIG. 10 illustrates a schematic of an artifact correction and image optimization process used in the automated analysis of intracoronary OCT frames, in accordance with one or more embodiments of the present disclosure.

### DETAILED DESCRIPTION OF THE DRAWINGS

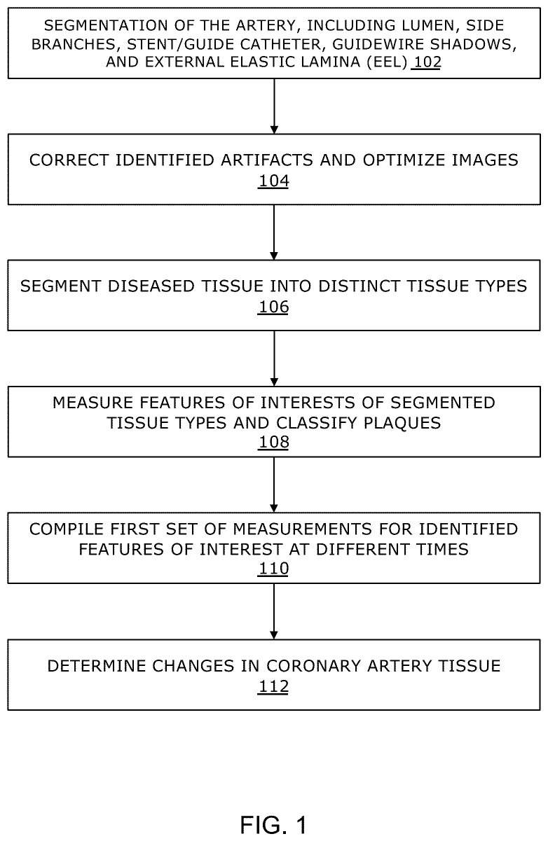

Referring to FIG. 1, illustrated is a flowchart of an automated computer-implemented method for analyzing a set of images of a coronary artery tissue, and which may be implemented using the system above. This method, structured for intracoronary optical coherence tomography (OCT) images, comprises several distinct stages, each addressing specific aspects of image analysis to evaluate the condition of the coronary artery effectively.

The process begins with the acquisition of OCT images, which are digital representations of the coronary artery tissue. These images serve as the input for the first neural network, a component of the method trained to segment the artery, including lumen, side branches, stent/guide catheter, guidewire shadows, and external elastic lamina (EEL), which are structures of the normal artery (lumen, side branches, EEL) or seen during OCT (stent/guide catheter, guidewire shadows).

In the present embodiments, the set of images comprises optical coherence tomography (OCT) images of the coronary artery tissue. In some non-limiting examples, the OCT images are pre-processed to normalize lighting conditions and contrast levels before analysis. OCT images, by their nature, can exhibit variations in lighting and contrast due to differences in imaging equipment, operator technique, and patient-specific factors. Such inconsistencies can pose challenges to automated analysis, as they may affect the visibility of tissue features and the detection of artifacts. To mitigate these issues, the OCT images are pre-processed to bring them to a standardized lighting and contrast level. Normalization of lighting conditions involves adjusting the brightness and illumination levels across the images so that all images have a uniform lighting environment. Similarly, contrast normalization adjusts the range of intensity values within the images to enhance the distinction between different tissue types and features. This pre-processing step is automated and integrated into the workflow of the method, ensuring that all OCT images are brought to a consistent quality standard before any detailed analysis is performed.

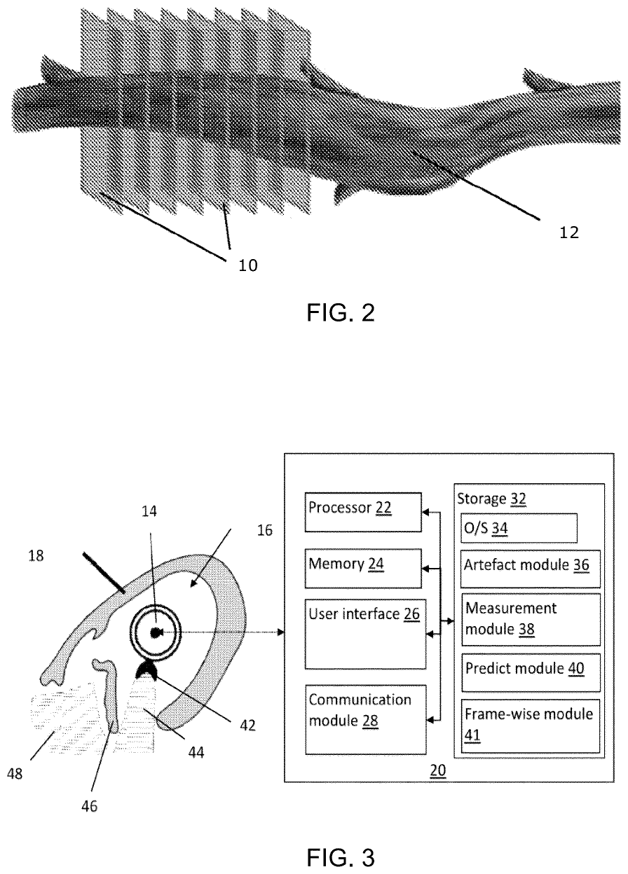

FIG. 2 illustrates an artery 12 from which a plurality of images 10 (also termed a set of images or image frames) have been captured. As shown, the images 10 are captured at regular intervals along the length of the artery 12. It will be appreciated that the number of images is merely illustrative and that the images may be captured along all or part of the length of the artery. The set may thus represent a whole or partial pullback along the length of the artery. Furthermore, the spacing between images may be varied as required.

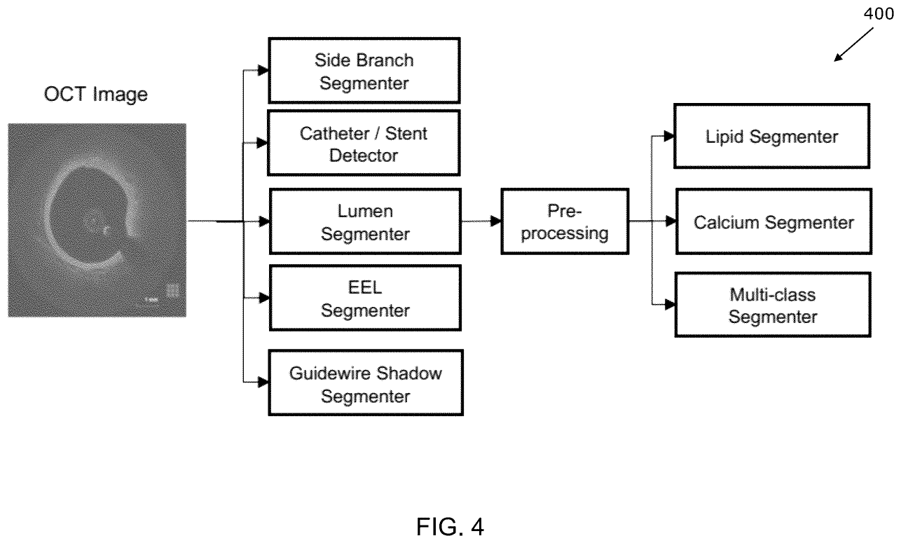

FIG. 3 schematically illustrates a system for capturing and processing an image captured from the artery. Such an image may be captured using an optical coherence tomography (OCT) device 14 which is inserted within the lumen 16 of the artery and images are captured as the device 14 is pulled back through the artery. For example, a pullback with a speed of 20 mm/s lasts about 2.5 seconds and allows imaging of about 72 mm of vessel. As explained in more detail below, the system may be used to analyze the thickness of the plaque region 18, which coats the inner wall of the artery. As schematically illustrated, atherosclerotic plaques are typically eccentric and vary in composition, which may make visualization of tissue features difficult. The OCT device 14 comprises a guide wire 42 which prevents OCT information being captured in the shadow region 44 of the guide wire 42. Furthermore, as explained in more detail below, there may be one or more artifacts 46 which also prevent OCT information from being captured in the shadow region 48 of the artifact. The OCT frames may be captured using standard techniques, e.g. using near-infrared light with wavelengths of between 1250 to 1350 nm, for example as described in “Consensus standards for acquisition, measurement, and reporting of intravascular optical coherence tomography studies: a report from the International Working Group for Intravascular Optical Coherence Tomography Standardization and Validation” by Tearney et al published in. J. Am. Coll. Cardiol. 59, 1058-1072 (2012).

The captured images may be processed or analyzed at a separate analysis device 20 which may be remote, e.g. in a different location to the OCT device 14 or may be at least partially local to the OCT device 14 (e.g. located adjacent a patient, or integrated into other imaging systems). Using a remote analysis device 20 may allow access to more powerful resources so that the raw data may be processed more quickly. A local processor has the advantage that it can be used in areas without any wireless connections. Accordingly, both local and remote processing may occur. The analysis device 20 may be implemented in hardware e.g. as a computing device such as a server or may be implemented in the cloud.

The analysis device 20 may comprise standard components such as a processor 22, a memory 24, a user interface 26, and a communication module 28. The memory 24 may be conventional memory which may include RAM and/ROM. The user interface 26 may be any standard interface, including a touch sensitive display screen, voice input, keyboard input etc. The communication module 28 may implement any suitable protocol, e.g. Wi-Fi, Bluetooth, or a wired connection.

The analysis device 20 further comprises storage 32 for storing modules which are to be carried out on the device. For example, the storage 32 may comprise an operating system module 34 to enable the device to operate. The storage 32 may also comprise an artifact module 36 which may be used to remove artifacts from the image as explained below. There may also be a measurement module 38 for determining or obtaining, for example, tissue arcs, areas or depths, and the interface between the plaque region (e.g. fibrous tissue layer) and the underlying layer as described below. There is also a predict module 40 which uses the measurements from the measurement module to make various predictions as described below. There may also be a frame-wise module 41 which is used to classify each frame as described below. Each of the artifact module 36, the measurement module 38, the predict module 40 and frame-wise module may be implemented as a neural network or other appropriate artificial intelligence system, and each is able to operate independently of the others where appropriate inputs are provided. In other words, the inputs need not be from the peer neural networks but may be other products or may produce independent useful final output.

The system in FIG. 3 may be considered a fully independent modular multi-metric auto-analysis system for OCT-based coronary atherosclerotic plaque analysis using deep neural networks. The four tools which are used to analyze the OCT images are the artifact module (may also be termed an artifact identifier), a tissue identification module, a measurement module, and a predict module (which may also be termed a presentation/event predictor). As described in more detail below, the input includes a whole artery pullback which is analyzed to generate micron-level measurement and context-free classification.

Referring back to FIG. 1, at step 102, the method includes for each image in the set of images, segmenting the image for artery features, including one or more of lumen, side branches and external elastic lamina (EEL), and OCT appearances, including one or more of stent, guide catheter and guidewire shadow, using a first neural network. The method employs the first neural network, which is a machine-learning model that has been trained on a vast dataset of OCT images. This training enables the neural network to recognize patterns and features within the images that are indicative of structures of normal arteries (lumen, side branches, EEL) or the OCT appearances (stent/guide catheter, guidewire shadows).

The neural network segments the lumen, which is the central blood-flow channel of the artery, for assessing the arterial diameter and potential blockages or narrowing. The neural network also identifies and segments side branches, which are smaller arteries that branch off from the main artery, for a detailed anatomical mapping and for assessing disease that may extend beyond the main arterial path. The neural network further segments the EEL, which defines the outer boundary of the tunica media (the middle layer of the artery wall), for evaluating the structural integrity of the artery and for measuring the thickness of the arterial wall.

In addition to anatomical features, the neural network segments OCT-specific appearances, which include artifacts and device-related structures to differentiate from actual arterial tissue. The neural network segments images to identify stents for evaluating their placement and interaction with the arterial wall. The neural network also segments images to identify guide catheters, which are used during the OCT procedure to guide the imaging device into place, helps differentiate these tool-related structures from arterial tissues. The neural network further segments images to identify shadows cast by the guidewire used during the OCT imaging, to ensure that they do not affect the analysis of the arterial structures.

At step 104, the method includes, when the image is segmented, correcting artifacts in the images by implementing an artifact correction methodology, using a second neural network. Artifacts, which can arise due to various imaging or environmental factors, may obscure or mimic disease characteristics, thus necessitating their identification for accurate analysis. For example, shadowing or blurring often arises due to technical limitations of the imaging process or patient movement during image acquisition, and can obscure the true nature of the tissue being examined or, in some cases, mimic the appearance of diseased tissue, leading to potential inaccuracies in diagnosis. Upon processing an image, the second neural network applies the artifact correction methodology, a subsequent step in the method that aims to enhance the image quality by mitigating or removing the artifacts. This ensures that the following stages of tissue segmentation and feature measurement are conducted on images that are as clear and accurate as possible, thereby enhancing the reliability of output methods.

In particular, the method includes correcting artifacts in the images using the artifact correction methodology followed by optimization (as discussed later). That is, once an image is classified by the second neural network as containing artifacts, the method progresses to artifact correction. This is important because artifacts, which are distortions or anomalies not part of the actual tissue structure, can significantly affect the accuracy of the tissue analysis. The artifact correction methodology employed is a systematic approach designed to mitigate or eliminate these distortions, thereby restoring the integrity of the image for accurate analysis of the coronary artery tissue. This involves several sub-steps designed to refine the images and eliminate or mitigate the artifacts, thereby enhancing the quality and reliability of the images for further analysis.

Herein, the artifact correction methodology further comprises optimizing image quality using an optimization procedure. The optimization procedure begins with the conversion of the OCT images to greyscale. This step standardizes the images by removing color variations, which simplifies the identification and correction of artifacts by focusing on variations in intensity rather than color. Following greyscale conversion, lumen masking is applied, which isolates the area within the coronary artery that is of clinical interest, effectively filtering out peripheral regions that are not relevant to the analysis. A polar transform is then applied to the lumen-masked images. This transformation reorients the images from a circular representation of the lumen to a rectangular one, facilitating easier comparison and analysis of the tissue around the entire circumference of the lumen. After the polar transform, the images are divided into panels, and the mean pixel intensity of each panel is measured. Histogram matching is subsequently performed, using the brightest panel as a reference to align the pixel intensity distributions across all panels both in 2D across a frame and in 3D between adjacent frames. This step ensures uniformity in the image's lighting and contrast, which is crucial for accurately identifying and measuring tissue features. The panels that have undergone histogram matching are then integrated into a single coherent image. To further enhance the clarity of the reconstructed image, a median filter is applied. Median filtering reduces noise in the image without blurring essential details, such as the edges of tissue structures, which are critical for accurate segmentation in later stages. Finally, adaptive filtering is employed, utilizing binary masks derived from thresholding operations. This step is designed to preserve essential features of the image while reducing the impact of outliers, ensuring that the tissue's true characteristics are maintained and clearly visible.

In the present embodiments, the second neural network is configured with machine learning algorithms, including supervised classification models, to identify artifacts within the set of images by analyzing patterns indicative of distortions. That is, the second neural network is specifically designed and trained using supervised techniques and the like, to recognize and classify these artifacts within the OCT images. In some examples, the second neural network may also be implemented for optimizing the image quality. Such optimization performed by the second neural network involves several key processes aimed at improving the clarity, detail, and usability of the OCT images. Such implementation may be contemplated by a person skilled in the art and thus not explained in detail herein for brevity of the present disclosure.

At step 106, the method includes, for the artifact corrected image, segmenting the diseased tissue into distinct tissue types using a third neural network, wherein the distinct tissue types include at least one of fibrous tissue, lipid-rich tissue, and calcific tissue. That is, with artifacts addressed, the method advances to the segmentation of diseased tissue into distinct types. This is accomplished by the third neural network, which segments the images to identify specific tissue types, such as fibrous, lipid-rich, and calcific tissues. This segmentation is critical for the detailed analysis of the tissue, allowing for a more nuanced understanding of the disease's nature and extent within the coronary artery. The third neural network is configured for differentiating among fibrous tissue, lipid-rich tissue, and calcific tissue, among potential others, each of which has specific implications for coronary artery disease diagnosis and management. Fibrous tissue typically represents areas of normal tissue, healed or healing injury in the artery wall and may indicate more stable plaque formation. Lipid-rich tissue, on the other hand, can signify areas of vulnerability within the artery wall, where plaque rupture could lead to acute coronary events. Calcific tissue is indicative of advanced atherosclerotic disease and can affect the mechanical properties of the vessel wall.

The third neural network is configured to implement a convolutional neural network (CNN) architecture for extracting and hierarchically organizing features from the set of images to segment the coronary artery tissue into the distinct tissue types, and classify plaque types. By applying this deep learning approach, the method achieves precise segmentation of the artery tissue. The third neural network is adapted for this segmentation by analyzing the pixel intensity, texture, and other image features that distinguish each tissue type. For example, calcific tissue may appear as brighter regions with sharply defined borders due to its higher density, while lipid-rich tissue may present as darker areas with diffuse borders. The network has been trained on a diverse set of OCT images annotated with these tissue types, allowing it to learn the complex patterns and features that define each. Once the third neural network processes an image, it outputs a segmented image where each pixel is classified as belonging to one of the tissue types of interest. This segmentation provides a detailed map of the tissue composition within the coronary artery, and, together with measurements below, can classify the type of plaque present.

In some embodiments, the method further includes implementing a combination of spatial filtering, intensity normalization, and edge detection techniques to facilitate tissue segmentation. Spatial filters are applied to the OCT images to reduce noise and smooth out variations that do not contribute to the essential tissue structure, which helps in highlighting the underlying patterns of the tissues without losing significant details. Different types of spatial filters can be applied, including Gaussian filters or median filters, each serving to enhance the homogeneity of the tissue regions while preserving the edges that demarcate different tissue types. Following spatial filtering, intensity normalization is performed on the OCT images. This process involves adjusting the intensity scale of the images to ensure a consistent dynamic range across the dataset. Edge detection algorithms are employed to identify the boundaries between different tissue types within the OCT images. These boundaries are used for segmenting the image into distinct tissue regions. Techniques such as the Sobel operator, Canny edge detector, or other gradient-based methods can be utilized to highlight the edges.

At step 108, the method includes identifying and measuring features of interests of the segmented tissue types, wherein the features of interests include one or more of arc, thickness, area, and depth for each tissue type. That is, after the diseased tissue within the coronary artery has been segmented into distinct tissue types such as fibrous, lipid-rich, and calcific tissues by the third neural network, the specific features of interest within the segmented tissue types are identified and measured. These features, which include, but are not limited to, arc, thickness, area, and depth of the tissues, are quantitatively assessed. The measurements derived from these features provide an evaluation of condition of the coronary plaque, including type of plaque and high-risk features. The thickness of a tissue segment, for instance, can be an indicator of plaque burden or the risk of plaque rupture, especially in the case of lipid-rich plaques covered by thin fibrous caps. The area covered by each tissue type within the cross-section of the artery provides insight into the extent of disease involvement and the progression of atherosclerotic plaque development. The depth of the tissue may provide details about the composition and stability of the plaque, and/or help guide management of the plaque, for example, additional drug or physical treatment or revascularization.

These measurements are performed using algorithms designed to accurately quantify the delineated features based on the segmented images. For example, edge detection algorithms may be employed to define the boundaries of a tissue segment accurately, enabling the precise calculation of its thickness and area. Texture analysis techniques can be utilized to assess the density of the tissues, distinguishing between the more homogeneous appearance of fibrous tissue and the heterogeneous appearance of lipid-rich regions. The identification and quantification of these features provide a comprehensive characterization of the plaque within the coronary artery, informing the clinician about the patient's current disease state. Further, these measurements can be used to monitor disease progression over time, especially when comparing baseline and follow-up OCT images. Changes in the arc, thickness, area, and depth of tissue types can indicate the impact of therapeutic interventions, such as drug or physical therapies, on plaque stabilization or regression.

In an embodiment, measuring the features of interest of the segmented tissue types comprises quantifying morphological and textural properties of each tissue type, including one or more of fibrous cap integrity, lipid pool heterogeneity, and calcification patterns, to provide an assessment of plaque composition. The assessment of morphological properties focuses on the structural aspects of the tissue types identified in the OCT images. For fibrous cap integrity, this involves measuring the thickness and continuity of the fibrous cap overlying atherosclerotic plaques. A thin or discontinuous fibrous cap is often associated with a higher risk of acute coronary events. In lipid pools within plaques, the method quantifies the variance in the size, shape, and distribution of lipid pools, as heterogeneous lipid-rich plaques are considered more unstable and prone to rupture. For calcific tissues, the method measures the patterns and distribution of calcification within the plaque, as certain calcification patterns may be associated with advanced atherosclerosis and influence the mechanical properties of the vessel wall. Textural analysis involves examining the pixel intensity patterns and variations within the segmented tissue regions to derive insights into the tissue's composition and structure. For example, the textural analysis can reveal the compactness of the fibrous cap or homogeneity of the lipid pool, providing further details about stability of the plaque.

At step 110, for sequential analysis, the method includes compiling a first set of measurements for each identified feature of interest from a first subset of images captured at a first time, and a second set of measurements for the same feature of interest from a second subset of images captured at a second time subsequent to the first time. Upon identifying and measuring the features of interest within the segmented tissue types from the OCT images, the method involves compiling these measurements to assess changes over time. This compilation is conducted for two distinct sets of images, including the first set, which are the baseline images, and are acquired at an initial time point; and the second set, which are follow-up images, and are captured at a subsequent time, allowing for a timeline analysis of condition of the coronary artery. For each identified feature of interest, such as the arc, thickness, area, and depth of the segmented tissue types, measurements are systematically recorded from the baseline images. These measurements serve as a reference point of state the coronary artery at the initial time of imaging. The same process is then repeated for the follow-up images, ensuring that measurements for the same features of interest are consistently obtained.

The method compiles these measurements into two distinct datasets, one representing the baseline condition and the other capturing the state of the coronary artery at the follow-up. This compilation facilitates a direct comparison between the two time points, revealing any changes that have occurred in the interim. By comparing the first set of measurements from the baseline images with the second set from the follow-up images, the method enables the detection of changes in the features of interest within the coronary artery tissue. These changes may manifest as variations in the thickness of fibrous caps, alterations in the area or arc covered by lipid-rich or calcific tissues, or shifts in the density of the tissue types. Such changes are indicative of the progression or regression of coronary artery disease extent or markers of stability and can be influenced by various factors, including natural disease progression, lifestyle changes, or the effect of therapeutic interventions.

In present embodiments, the first and second subsets of images are aligned and co-registered using anatomical or fiduciary landmarks within the coronary artery tissue. Anatomical landmarks within the coronary artery, such as bifurcations, side branches, or specific plaque formations, serve as reference points for alignment. These landmarks are identifiable features that remain generally consistent across different imaging sessions. The process of alignment begins by identifying these landmarks in both subsets of images. Once identified, image processing algorithms are employed to adjust and align the images. This step may involve transformations such as translation, rotation, and scaling to achieve precise alignment. Following the alignment, co-registration is performed, which involves overlaying the aligned images to achieve a match of the anatomical landmarks.

At step 112, the method includes determining, using the compiled first and second sets of measurements, changes in the coronary artery tissue, wherein the changes are indicative of progression or regression of a diseased state, to be utilized for determining an efficacy of drug or device therapy, or, using a single set of images, prediction of multiple adverse cardiovascular events (MACE) such as cardiac death or myocardial infarction to help guide treatment. Utilizing the quantitative data obtained from the two distinct time points, the method determines the changes that have occurred in the coronary artery tissue. These changes are analyzed to determine whether they represent a progression or regression of the diseased state within the artery. The comparison between the first set of measurements from the baseline images and the second set from the follow-up images may reveal any alterations in the features of interest, such as changes in the thickness of fibrous caps, the arc or area covered by lipid-rich or calcific tissues, or the density of these tissues. An increase in fibrous cap thickness or a reduction in the area or are covered by lipid-rich tissue, for example, might indicate a positive response to therapy, suggesting plaque stabilization or regression. Conversely, a decrease in fibrous cap thickness or an expansion of lipid-rich areas could signal disease progression.

In some embodiments, the method involves applying a fourth neural network to analyze the compiled first and second sets of measurements for determining the efficacy of drug or device therapy, wherein the fourth neural network utilizes a predictive model trained on historical data correlating tissue characteristics with patient outcomes, including myocardial infarction and cardiac death. The fourth neural network specifically focuses on the analytical task of interpreting the compiled measurements from the baseline and follow-up sets of OCT images, to determine any impact on the coronary artery tissue by observing changes in the measured features over time. The fourth neural network operates on a predictive model that has been meticulously trained on a comprehensive dataset of historical OCT images and associated patient outcomes. This training dataset includes diverse examples of tissue characteristics, such as the thickness of fibrous caps, the arc or area covered by lipid-rich plaques, and the density of calcific tissues, alongside the corresponding patient outcomes, which may range from stable conditions to events like myocardial infarction. By analyzing the first and second sets of compiled measurements, the fourth neural network assesses the progression or regression of tissue characteristics indicative of coronary artery disease. For instance, a decrease in lipid-rich plaque area or an increase in fibrous cap thickness in the follow-up measurements compared to the baseline may suggest plaque stabilization, potentially indicating the effectiveness of a particular drug therapy. The predictive model within the fourth neural network leverages this historical data to draw correlations between specific changes in tissue characteristics and the likelihood of various patient outcomes. This approach enables the fourth neural network to predict the therapeutic efficacy based on the observed changes in tissue features over time.

By quantifying these changes, the method allows for evaluating the impact of drug or physical therapy, for instance, pharmacological treatments like statins, or device therapy such as stent placement. The ability to measure the efficacy of a given therapy on the condition of the coronary artery helps in managing coronary artery disease, and allows for a tailored approach to treatment, where therapeutic strategies can be adjusted based on the patient's individual response over time. Furthermore, the insights gained from the analysis of changes in coronary artery tissue can guide clinical research and the development of new therapies. By establishing a clear correlation between specific treatment regimens and quantifiable changes in artery tissue characteristics, the method can contribute to evidence-based recommendations for disease management.

In addition, in present embodiments, each image is of the coronary artery tissue of a patient, and wherein the method comprises determining, using the measurement of each identified feature of interest, a likelihood of the patient having a particular manifestation of a coronary artery disease. This determination involves a detailed analysis of the quantified features, such as the thickness of fibrous caps, the arc or area covered by lipid-rich tissues, the density and patterns of calcification, among others. The method utilizes the measurements of these features to assess the patient's coronary artery condition, which is then used to estimate the probability of the patient having an event over a defined time period. Advanced statistical or machine learning algorithms may be employed to analyze the data. These algorithms can compare the patient's data against known patterns and thresholds derived from clinical research and historical patient data to estimate the likelihood of specific disease manifestations.

In implementation of the present disclosure, the coronary artery disease manifestations are cardiac death or myocardial infarction and the identified features of interest are fibrous tissue thickness or lipid. This specific feature of interest, i.e., the thickness of the fibrous cap covering a lipid containing atherosclerotic plaque, is evaluated due to their established correlation with the risk of myocardial infarction and cardiac death. Once the OCT images are segmented to identify the lipid and fibrous tissue regions, the method employs algorithms to measure the arcs and thickness of these regions accurately. This involves delineating the boundaries of the arc and fibrous caps and quantifying the distance between these cap boundaries across various segments of the plaque.

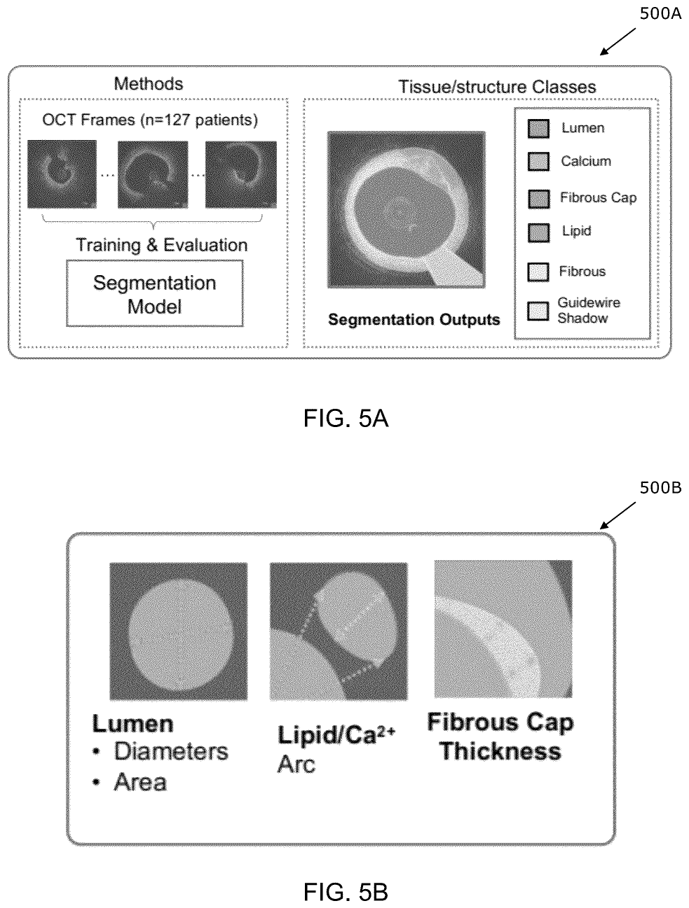

Referring to FIG. 4, illustrated is an outline of a deep learning model (as represented by reference numeral 400) used in the automated method for processing and analyzing intracoronary optical coherence tomography (OCT) images. The model includes various components that contribute to the identification and segmentation of specific structures within the coronary artery. An OCT image serves as the input for the model. The image first goes through a series of segmenters that are specialized in recognizing different anatomical features and artifacts present within the coronary artery. A side branch segmenter is designed to identify and segment the side branches of the coronary artery, which are anatomical landmarks for image registration and analysis. The catheter/stent detector is responsible for detecting the presence of guide catheters or stents, which may introduce artifacts into the image. The lumen segmenter focuses on the internal space of the coronary artery, defining the lumen boundary, which is crucial for assessing conditions like stenosis. The EEL (External Elastic Lamina) segmenter identifies the external boundary of the artery, providing measurements that are essential for understanding the artery's overall structure and the extent of atherosclerotic disease. Additionally, the guidewire shadow segmenter is tasked with identifying and accounting for artifacts caused by the shadow of the guidewire used during the OCT procedure.

The images then go through pre-processing, a step where artifacts such as noise or distortions that could affect the accuracy of the analysis are identified and removed, and the image optimized. The pre-processed image is then passed to the lipid segmenter and calcium segmenter, which identify and segment regions within the artery containing lipid and calcium deposits, respectively. These segments are indicative of atherosclerotic plaques and their composition, and their precise identification helps in disease assessment and management. Finally, a multi-class segmenter is employed to categorize the various types of tissue present in the plaque and artery, integrating the information provided by all prior segmenters. This segmentation approach facilitates a comprehensive analysis of the plaque components and the overall structural integrity of the coronary artery.

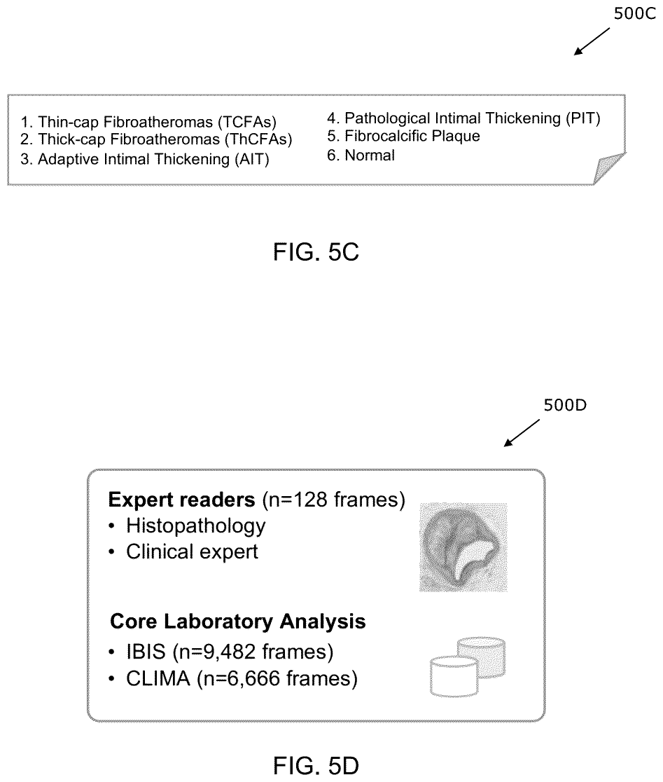

Referring to FIGS. 5A-5D, in combination, illustrated are stages involved in the automated computer-implemented method for analyzing intracoronary optical coherence tomography (OCT) images for assessing coronary artery disease. As shown, the process is subdivided into four primary components, each depicting a different aspect of the method. FIG. 5A illustrates a pattern recognition stage (as represented by reference numeral 500A) which begins with the input of OCT frames from patients. These images are subject to an automated segmentation model, which employs machine learning techniques to identify and segment the coronary artery tissue into different classes based on their distinct characteristics. The segmentation model is trained and evaluated using a set of OCT frames, and the outputs define various tissue/structure classes such as the lumen, calcium deposits, fibrous cap, lipid regions, fibrous tissue, and areas affected by the guidewire shadow. FIG. 5B illustrates a measurement stage (as represented by reference numeral 500B) which involves measurement of identified features of interest within the segmented tissue classes. This includes quantifying the lumen diameter and area, the arc, area and depth of lipid/calcium deposits, and the thickness of the fibrous cap. These measurements characterize the condition of the coronary artery and are used to evaluate the presence, extent, and type of coronary artery disease. FIG. 5C illustrates a plaque classification stage (as represented by reference numeral 500C) which involves categorizing the plaques identified in the OCT images into various types. This classification is based on the morphological and textural properties of the plaques, such as thin-cap fibroatheromas (TCFAs), thick-cap fibroatheromas (ThCFAs), adaptive intimal thickening (AIT), pathological intimal thickening (PIT), fibrocalcific plaque, and normal tissue state, which aids in the assessment of plaque composition, the determination of disease severity, and the likelihood of specific manifestations of coronary artery disease. FIG. 5D illustrates an external validation stage (as represented by reference numeral 500D) which involves the validation of outputs of the method against expert readings for histopathology and clinical scans and core laboratory analysis for 2 clinical trials. Expert readers, including histopathologists and clinical experts, review a subset of frames to validate the plaque classifications and measurements. Additionally, core laboratory analysis of intravascular OCT from the IBIS-4 and CLIMA trials further corroborates the accuracy of the outputs of the method. This external validation serves to ensure that the results are consistent with established clinical and histopathological standards, ensuring the reliability of the method for clinical use.

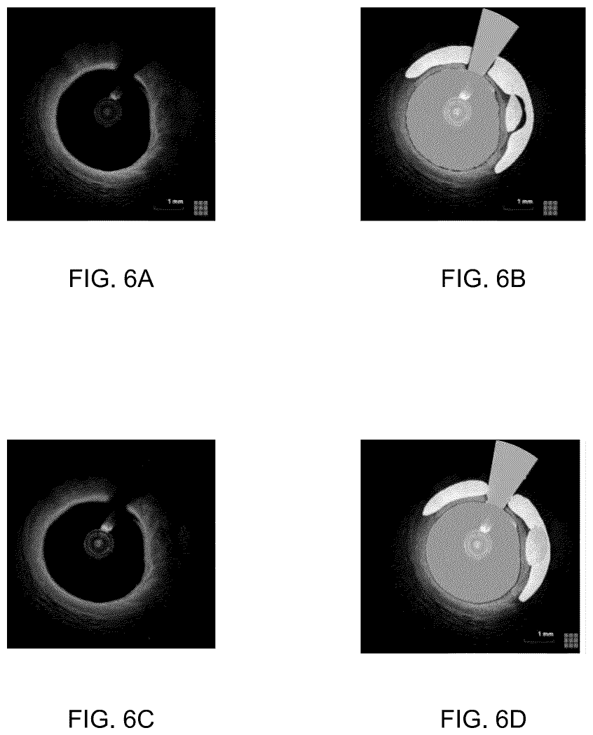

Referring to FIGS. 6A-6D, depicted are results of the segmentation process applied in the automated analysis of intracoronary optical coherence tomography (OCT) images. FIG. 6A depicts a first image which is an original OCT image of the coronary artery tissue. This raw image serves as the starting point for the analysis and represents the artery without any modification or annotation. FIG. 6B depicts a second image which represents a manually annotated OCT image (ground truth). This image serves as a reference standard for segmentation, and shows the coronary artery tissue segmented into different classes with distinct colors indicating various tissue and structure types. The colors may correspond to different tissue types such as calcifications, lipid regions, and fibrous tissue, which are features of interest for analyzing the coronary artery. FIG. 6C depicts a third image which is the OCT image post-artifact correction and optimization. The artifact correction methodology has been applied to this image to remove any distortions that could affect the analysis. This image is then used for automated segmentation. FIG. 6D depicts a fourth image which is the corrected image shown with superimposed colored regions indicating prediction of the model of different tissue types within the coronary artery.

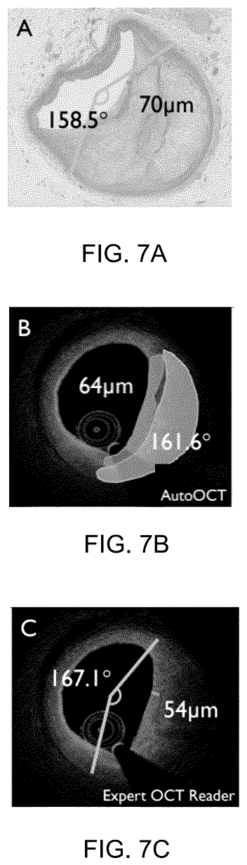

Referring to FIGS. 7A-7C, illustrated is an overview of the performance of the automated method for analyzing intracoronary optical coherence tomography (OCT) images in lipid-containing lesions, highlighting the comparison of fibrous cap thickness and lipid arc measurements as assessed by the automated system and by an expert OCT reader. FIG. 7A depicts a histology section illustrating the measurement of fibrous cap thickness (FCT) and lipid arc within a plaque. FIG. 7B depicts an OCT image processed by the automated method, where measurements of the fibrous cap thickness and the angle of the lipid arc are annotated. FIG. 7C depicts the same OCT frame with annotations made by an expert OCT reader for comparison purposes. It may be understood that the FCT and lipid arc measurements are key factors in evaluating the vulnerability of a plaque and the potential risk for coronary events. The measurements are denoted in microns for thickness and degrees for the arc, offering a precise quantification of these critical features.

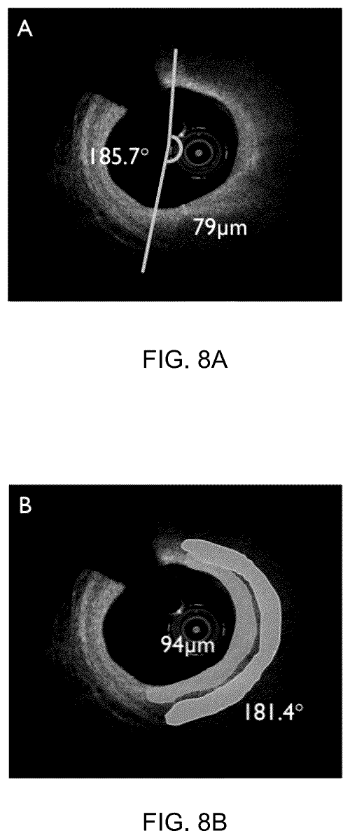

Referring to FIGS. 8A-8B, illustrated is an overview of the performance of the automated method in measuring features of interest within the coronary artery tissue, particularly focusing on fibrous cap thickness and lipid arc in fibroatheroma. FIGS. 8A-8B provides a comparative analysis of the measurements obtained by a core laboratory versus those acquired by the automated analysis method. FIG. 8A depicts an OCT image annotated by the core laboratory, displaying measurements of fibrous cap thickness and the angle of the lipid arc, which are indicators of plaque vulnerability. FIG. 8B depicts the same anatomical features identified and measured by the automated method within the OCT image. These annotations are used for assessing the composition and potential risk associated with plaques in the coronary artery.

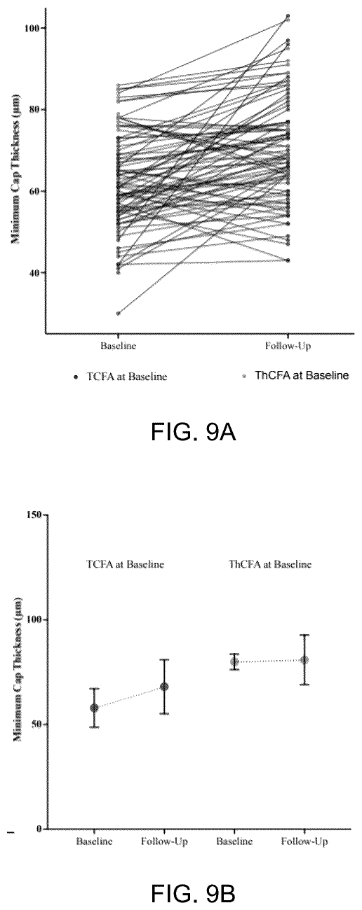

FIGS. 9A and 9B present graphical representation that illustrates the changes in the minimum thickness of the fibrous cap of coronary artery plaques over time, as determined by the present method. FIG. 9A presents a line graph where individual data points represent the minimum fibrous cap thickness for individual fibroatheroma lesions at two different time points: baseline and follow-up following drug treatment. Each line connects the measurement of a single lesion at baseline to its measurement at follow-up, with different markers used to denote thin-cap fibroatheromas (TCFAs) at baseline versus thick-cap fibroatheromas (ThCFAs) at baseline. This illustrates the changes in fibrous cap thickness for each lesion between the two time points, providing insight into the progression or regression of the disease. FIG. 9B presents a bar graph with error bars showing the mean minimum fibrous cap thickness at baseline and follow-up for two distinct groups: lesions initially identified as TCFAs and those identified as ThCFAs. The error bars indicate the variability within each group. Thereby, FIGS. 9A and 9B demonstrate capability of the present method to measure a key feature of interest, the fibrous cap thickness, and to monitor changes in this feature over time.

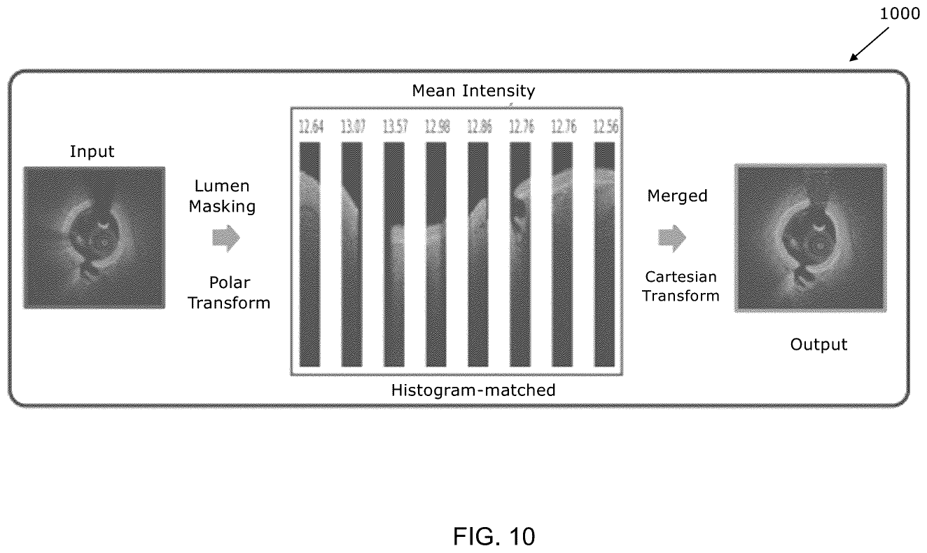

FIG. 10 schematically illustrates an artifact correction and image optimization process (as represented by reference numeral 1000) used in the automated analysis of intracoronary optical coherence tomography (OCT) frames, which enhances the quality of the input images. The process begins with the ‘Input’ stage, where the OCT frame is displayed as it appears before any processing. This image is first converted to greyscale to standardize the pixel intensity, which allows for more consistent analysis across the set of images. Following this conversion, lumen masking is applied to isolate the coronary artery lumen from the surrounding tissue, focusing on the area of interest. A polar transform is then performed from the center point of the lumen, converting the circular representation into a flat, unwrapped image. This transformation simplifies the comparison of features along the circumference of the coronary artery.

Further, the process involves the greyscale image split into panels with measured mean pixel intensities indicated above each panel. These panels are subjected to histogram matching, a process that aligns the pixel intensity profiles across the panels using the brightest panel as a reference, using matching in 2D within the same frame, and 3D between adjacent frames. This ensures uniform lighting and contrast across the image. The ‘Output’ image shows the reconstructed OCT image after a Cartesian transform is applied to revert the unwrapped image back to its original circular format. This image reflects the combined data from the histogram-matched panels and is normalized for intensity, providing a clear and artifact-corrected version of the original input.

The objective of the present disclosure is to determine if artificial intelligence (AI)-based analysis of intracoronary optical coherence tomography (OCT) can identify features that predict drug success/failure and future multiple adverse cardiovascular events (MACE). Intracoronary OCT can identify changes due to drugs and high-risk plaques causing future patient events, but analysis requires expert clinician or core laboratory interpretation, while artifacts and limited sampling markedly impair reproducibility. The present system is an AI-based system to rapidly process, optimize and analyze OCT images, and identify changes in plaque composition and high-risk features. The AI modules are designed to correct poor quality or artifact-containing OCT images, identify tissue and plaque composition, and measure multiple parameters including lumen area, lipid and calcium arcs, and fibrous cap thickness (FCT), with outputs comprising segmented images, plaque classification, and clinically useful (including high-risk) parameters. The present system demonstrates that unbiased automatic measurement of intracoronary OCT images is feasible, and can identify changes in plaque structure associated with stabilization and patient events. The present system may be a valuable tool for both clinical trials of drug efficacy and identification of high-risk plaques to help guide patient management.

### Experimental Data

127 pullbacks (36,212 frames) from 106 patients were used for model development, and ex-vivo OCT pullbacks from post-mortem arteries to validate tissue classification. External validation against core laboratory analysis was performed using 83 baseline and follow-up patients from the IBIS-4 high-intensity statin (HIS) study to identify features indicating plaque stabilization, and 62 patients from the natural history CLIMA study to predict MACE. The present system could recover images containing common artifacts. The derived plaque classification correlated well with histology (diagnostic accuracy 83.6%). The system replicated IBIS-4 core laboratory changes in plaque composition after 13 m of HIS treatment, including reduced lesion lipid arc (13.3° vs. 12.5°, p<0.001) and increased minimum FCT (18.9 μm vs. 24.4 μm, p=<0.001). The system also identified similar high-risk plaques leading to future MACE to the CLIMA core laboratory. Thus, the system-based analysis of whole coronary artery OCT identifies and measures features that correlate with plaque stabilization and high-risk plaques. AI-based OCT analysis may augment clinician or core laboratory analysis of intracoronary OCT images for both clinical trials of drug efficacy and identifying high-risk lesions.

To develop the present system, we analyzed 127 complete OCT pullbacks from 106 unselected patients with coronary artery disease (CAD) from three UK cardiothoracic centers (Papworth (Cambridge), Swansea, and St Peter's Hospital Chertsey, UK), totaling 36,212 OCT frames. All patients enrolled in clinical studies provided informed consent, and all pullbacks were included for analysis with no exclusion criteria. Histopathological validation used a dataset of co-registered OCT and histology from 13 post-mortem pullbacks from left anterior descending arteries with written consent from relatives. External validation used 83 patients from the OCT arm of the Integrated Biomarker Imaging Study-4 (IBIS-4, NCT00962416), where non-culprit arteries of ST-elevation myocardial infarction (STEMI) patients were imaged at baseline and after 13 months of high-dose Rosuvastatin treatment. 62 patients were analyzed from the CLIMA study (NCT02883088), a prospective observational, multi-center registry recruiting 1003 consecutive patients undergoing OCT assessment of proximal LAD atherosclerosis by OCT. All pullbacks were acquired with frequency-domain OCT systems using C7-XR™ or OPTIS™ (Abbott Vascular, Santa Clara, CA, USA) using a non-occlusive technique.

The system's software was developed in Python (3.8) with training facilitated using the University of Cambridge high performance computing cluster (Wilkes3). Segmentation masks for different artery structures and plaque components were extracted using a DeepLabv3+ deep learning convolutional neural network (CNN) architecture. The model was trained with annotated frames in axial cross-sections after greyscale conversion, with a spatial size of (512, 512). Data were randomly divided into training, testing and validation sets in a 14:1:1 patient level ratio respectively, strictly avoiding data repetition. A hybrid loss function comprising cross-entropy and Dice loss was used for training, and the adaptive moment estimation (ADAM) optimizer for the segmentation model, with initial learning rate, drop factor, and drop period set empirically to 0.001, 0.1, and 10, respectively. A custom-designed data loader was used to overcome imbalance and data pre-processed and optimized prior to being used in training. Extensive ablation studies aided selection of the best model architecture.

OCT pullbacks were exported in DICOM (Digital Imaging and Communications in Medicine) format for offline analysis using a LightLab Imaging workstation (St. Jude Medical). Manual segmentation of frames was performed using The Medical Imaging Interaction Toolkit (v 2021.10) software. All ground-truth annotation was performed in axial cross-sections by an experienced intravascular imaging specialist following accepted plaque definitions. All frames were labeled, regardless of classification, data quality or presence of imaging artifacts, but excluding frames within the guide catheter or stents which were noted using binary labels. Lumen contours, bifurcations, and the external elastic lamina (EEL) were defined, with structures in-between classified as guidewire shadow, bifurcations, or as plaque components. Plaques and tissues were classified using standard definitions: Plaques were defined as a mass lesion within the arterial wall with loss of the normal tissue tri-layer appearance or focal intimal thickening, calcification as a signal-poor area with sharply delineated borders and low attenuation, lipid as a signal-poor region with poorly defined borders with fast OCT signal drop-off, and fibrous cap as a fibrous layer overlying lipid/necrotic core or calcium. Normal vessel and fibrous tissue were annotated as one structure, but each plaque component was annotated separately.

Left anterior descending arteries from 13 donors underwent OCT imaging post-mortem before co-registration with histopathology as described previously and in the Supplement. OCT pullbacks were analyzed for plaque classification and lumen, EEL, and plaque parameter measurements by the system and an expert interventional cardiologist with >10 year's experience (total intravascular imaging experience: >500 IVUS and >500 OCT procedures), blinded to the system's results. Histological plaque classification and measurements were validated by an independent experienced cardiac pathologist, blinded to coronary imaging and the system.

Continuous variables are summarized as mean±SD and categorical variables as counts (percentage). Agreement between imaging measurements (manual or by the system) or with histology was compared using intraclass correlation coefficients (ICC) for absolute agreement and Bland-Altman plots comparing mean against difference in measurements. p values were reported for exploratory purposes for model performance in against clinical studies without any claims of significance. Student's t- and χ2 tests were applied when appropriate. Two-sided p values are reported throughout adopting 0.05 as significant. Analyses were performed using SPSS 28.0.0 (SPSS Inc, IBM Computing) and R version 3.4.0 (R Foundation for Statistical, Vienna, Austria).

Overall 366 pullbacks from 297 patients were analyzed, representing 58,840 OCT frames. Separate datasets were used for training (106 patients, 106 pullbacks, 36,212 frames) comprising data from unselected patients from three UK cardiothoracic centers, histopathological validation (13 patients, 24 pullbacks, 6,480 frames), and external validation (145 patients, 236 pullbacks, 16,148 frames) from IBIS-4 and CLIMA studies. Post-mortem donors were aged 47-85 years, 71.4% male, and included both cardiovascular and noncardiovascular causes of death (Table S1). IBIS-4 and CLIMA patient characteristics are described in their respective publications.

TABLE S1
Demographics of post-mortem donors

| | Overall (n = 14) | | CV Death (n = 8) | | Non-CV Death (n = 6) | |
|---|---|---|---|---|---|---|
| Male, n (%) | 10 | (71.4) | 8 | (100.0) | 2 | (33.3) |
| Age, y (SD) | 71.1 | (11.8) | 76.8 | (9.8) | 63.6 | (10.3) |

Comorbidities, n (%)

| | | | | | | |
|---|---|---|---|---|---|---|
| Ischaemic Heart Disease | 7 | (50.0) | 6 | (75.0) | 1 | (16.7) |
| Cerebrovascular Disease | 2 | (14.3) | 1 | (12.5) | 1 | (16.7) |
| Extra-cardiac Arteriopathy | 6 | (42.9) | 4 | (50.0) | 2 | (33.3) |
| Diabetes Mellitus | 1 | (7.1) | 1 | (12.5) | 0 | (0.0) |
| Hypertension | 5 | (35.7) | 3 | (37.5) | 2 | (33.3) |
| Cardiac Failure | 6 | (42.9) | 4 | (50.0) | 2 | (33.3) |
| CTEPH | 3 | (21.4) | 1 | (12.5) | 2 | (33.3) |

CV indicates cardiovascular; CTEPH indicates chronic thromboembolic pulmonary hypertension

128 unique OCT frames were analyzed by the present system and successfully co-registered with their corresponding histological sections. By histology, lesions were classified as normal vessel (n=3, 2.3%), adaptive intimal thickening (AIT)(n=19, 14.8%), pathological intimal thickening (PIT)(n=29, 22.7%), fibrocalcific (n=8, 6.3%), and fibroatheroma (n=69, 53.9%). 22 (17.2%) fibroatheromas were thin cap fibroatheromas with 47 fibroatheroma defined as thick cap fibroatheromas.

We analyzed lumen parameters (area, minimum and maximum diameter), lipid and calcium arcs, and minimum FCT in plaques with different classification (Table 1). Although comparison with histology requires perfect pressure fixation, preparation of sections, and accurate co-registration, there were no significant differences in any parameter for any plaque type between histology and the present system. In particular, the higher risk features of mean minimum FCT and lipid arc in TCFA measured by the present system were similar to histology (48.9±15.5 μm vs. 54.4±14.5 μm, p=0.905, and 185.0±71.4° vs. 164.5±69.7°, p=0.644)(Table 1). Co-registered OCT frames were also classified by the present system utilizing standardized tissue definitions (10, 36). The overall diagnostic accuracy of the present system for lipid and calcium tissue across all lesions was 69.5% and 68.0% respectively, and for plaque classification was 72-86% for different lesions, and 83.6% for TCFA (Table 2). Interestingly, 4/14 incorrectly classified TCFA had a lipid arc extension <90° suggesting discord between the histopathologic and OCT definition of fibroatheroma.

Post-mortem OCT pullbacks were also analyzed by an expert interventional cardiologist, and compared to histopathology and the present system. Although there were differences in lumen area and lipid arc between the expert reader and histopathology (Table 1), the diagnostic accuracy of plaque classification was between 65.6%-89.1% (Table 2). Overall measurements made with the present system showed excellent correlation with expert reader (ICCa 0.861 [95% CI 0.841-0.879, p=<0.001]), although differences were present with individual features. For example, lumen area, minimum lumen diameter, and maximum lumen diameter correlated well for lipid containing lesions (ThCFA, TCFA, and fibrocalcific plaque)(mean differences 0.23 mm² (p=0.061), 0.07 mm (p=0.004), and 0.01 mm (p=0.920) respectively. Lipid and calcium arc measurements differed (mean difference 40.0°, p=<0.001, and 40.5°, p=0.002, respectively), but minimum FCT measurements <200 microns were similar (mean difference 2.2 μm, (p=0.427). The present system correlation with expert reader was also good for TCFAs. Lumen area, minimum lumen diameter, and maximum lumen diameter were similar (mean differences 0.06 mm² (p=0.605), 0.01 mm (p=0.948), and 0.04 mm (p=0.740) respectively). Mean difference in FCT was 0.00 μm (p=1.000), and although lipid arc measurements differed (mean difference 119.8°, p=0.007), the present system measurements were closer and not significantly different to ground-truth histology measurements compared with expert reader (Present system 13.1°[p=0.469], expert reader 97.2° [p=0.002]).

TABLE 1
Histological, Present System, and optical coherence tomography features for each plaque subtype

| Histological Classification | | | | | |
|---|---|---|---|---|---|
| AIT | PIT | ThCFA | TCFA | Fibrocalcific | |
| (n = 19) | (n = 29) | (n = 47) | (n = 22) | (n = 8) | P-value |

Histology

| | | | | | |
|---|---|---|---|---|---|
| Lumen Area, (mm²) | 1.44 ± 0.85 | 3.13 ± 2.47 | 4.99 ± 3.20 | 4.56 ± 3.33 | 4.44 ± 2.25 |
| Min Lumen Diam, (mm) | 0.94 ± 0.41 | 1.31 ± 0.54 | 1.95 ± 0.77 | 1.66 ± 0.67 | 1.86 ± 0.51 |
| Max Lumen Diam | 1.91 ± 0.41 | 2.69 ± 1.02 | 3.06 ± 1.12 | 3.10 ± 1.43 | 2.90 ± 0.65 |
| Lipid Arc, (°) | n/a | n/a | 144.3 ± 54.3 | 164.5 ± 69.7 | 134.9 ± 80.3 |
| MinFCT, (μm) | n/a | n/a | 113.7 ± 37.7 | 54.4 ± 14.5 | 109.6 ± 47.8 |
| Calcium Arc, (°) | n/a | n/a | 60.6 ± 41.0 | 100.7 ± 25.7 | 73.0 ± 69.8 |

AutoOCT

| | | | | | | |
|---|---|---|---|---|---|---|
| Lumen Area, (mm²) | 2.84 ± 0.99 | 5.02 ± 3.29 | 4.86 ± 2.81 | 4.95 ± 3.20 | 4.86 ± 2.64 | 0.341 |
| Min Lumen Diam, (mm) | 1.64 ± 0.28 | 2.08 ± 0.64 | 2.01 ± 0.64 | 2.05 ± 0.70 | 2.12 ± 0.71 | 0.238 |
| Max Lumen Diam | 2.22 ± 0.32 | 2.84 ± 0.87 | 2.88 ± 0.83 | 2.82 ± 0.87 | 2.73 ± 0.60 | 0.107 |
| Lipid Arc, (°) | n/a | n/a | 161.1 ± 58.8 | 185.0 ± 71.4 | 158.1 ± 60.3 | 0.644 |
| MinFCT, (μm) | n/a | n/a | 180.4 ± 82.8 | 48.9 ± 15.5 | 72.8 ± 53.4 | 0.905 |
| Calcium Arc, (°) | n/a | n/a | 49.4 ± 13.2 | 56.5 ± 19.9 | 73.2 ± 52.9 | 0.212 |

Expert OCT Reader

| | | | | | | |
|---|---|---|---|---|---|---|
| Lumen Area, (mm²) | 3.07 ± 0.97 | 3.53 ± 1.10 | 5.59 ± 3.15 | 4.07 ± 2.80 | 4.41 ± 1.13 | 0.031 |
| Min Lumen Diam, (mm) | 1.70 ± 0.22 | 1.91 ± 0.37 | 2.26 ± 0.70 | 1.69 ± 0.62 | 2.26 ± 0.22 | 0.523 |
| Max Lumen Diam | 2.29 ± 0.46 | 2.42 ± 0.36 | 2.90 ± 0.80 | 2.69 ± 0.68 | 2.53 ± 0.47 | 0.326 |
| Lipid Arc, (°) | n/a | n/a | 241.1 ± 88.5 | 295.6 ± 74.0 | n/a | 0.024 |
| MinFCT, (μm) | n/a | n/a | 198.8 ± 104.9 | 42.7 ± 13.8 | n/a | 0.383 |
| Calcium Arc, (°) | n/a | n/a | 44.4 ± 10.4 | 66.6 ± 11.1 | 152.3 ± 88.6 | 0.509 |

AIT indicates adaptive intimal thickening;
FCT, fibrous cap thickness;
PIT, pathological intimal thickening;
TCFA, thin-cap fibroatheroma;
and ThCFA, thick-cap fibroatheroma (data presented are mean ± SD, p-value against histology for TCFA plaque classification shown)

TABLE 2
Accuracy of Present System and optical coherence tomographic
plaque classification compared with histology

| | Histological Classification | | | | |
|---|---|---|---|---|---|
| AutoOCT | AIT | PIT | ThCFA | TCFA | Fibrocalcific |
| Sensitivity, (%) | 57.9% | 37.9% | 61.7% | 36.4% | 12.5% |
| Specificity, (%) | 90.8% | 82.8% | 77.8% | 93.4% | 90.8% |
| PPV, (%) | 52.3% | 39.4% | 61.7% | 53.4% | 8.3% |
| NPV, (%) | 92.6% | 82.0% | 77.8% | 87.6% | 94.0% |
| Diagnostic Accuracy, (%) | 85.9% | 72.7% | 71.9% | 83.6% | 85.9% |

Expert OCT Reader

| | | | | | |
|---|---|---|---|---|---|
| Sensitivity, (%) | 52.6% | 41.4% | 53.2% | 31.8% | 50.0% |
| Specificity, (%) | 93.6% | 89.9% | 72.8% | 84.0% | 91.7% |
| PPV, (%) | 58.7% | 54.6% | 53.2% | 29.2% | 28.6% |
| NPV, (%) | 91.9% | 83.9% | 72.9% | 85.6% | 96.5% |
| Diagnostic Accuracy, (%) | 87.5% | 78.9% | 65.6% | 75.0% | 89.1% |

AIT indicates adaptive intimal thickening; PIT, pathological intimal thickening; TCFA, thin-cap fibroatheroma; and ThCFA, thick-cap fibroatheroma; PPV, positive predictive value; NPV, negative predictive value.

Although these results indicate generally good correlation between the present system and both histology and an expert reader, we undertook stricter validation against external expert core laboratories using frame-based comparison from clinical trials. The OCT sub-study of IBIS-4 showed that high-intensity statin treatment increases minimum FCT, reduces mean lipid arc and alters % of frames showing different lesion types (Table 3) consistent with stabilization of lesions. Serial OCT imaging was available from 83 patients (153 arteries) for lesion type, and 31 arteries from 27 patients had fibroatheromas (ThCFA or TCFA) at both time points. The present system minimum FCT correlated well with core laboratory measurements (ICCa 0.659 (95% CI 0.620-0.695), p=<0.001), with further analysis showing a non-significant and sub-pixel-level average difference-3.1 μm (p=0.241). The present system lipid arc also demonstrated good correlation with core laboratory measurements (ICCa 0.750 (95% CI 0.682-0.801), p=<0.001), with further analysis showing a clinically acceptable difference of only 18.3 degrees (p=<0.001). The present system minimum FCT over whole vessel length increased from 62.9±28.4 to 81.8±33.4 (p=<0.001), similar to core laboratory analysis (64.88±19.89 to 87.88±38.08, p=0.008). The present system mean lipid arc over the whole vessel decreased from 63.1±21.7 to 49.8±20.3 (p=<0.001), again similar to the core laboratory (55.94±31.04 to 43.46±3.48, p=0.013)(Table 3). Both the present system and core laboratory found no change in % normal vessel frames and an increase in fibrocalcific plaques, although the present system found increased % fibrous tissue frames while the core laboratory found a decrease (Table 3).

TABLE 3
Serial Vessel-Level OCT Analyses

| | Number of | Number of | | | Mean Change | | |
|---|---|---|---|---|---|---|---|
| | Patients | Vessels | Baseline | Follow-up | (95% CI) | | P-value |
| IBIS-4 | | | | | | | |
| ROI Length, mm | 83 | 153 | 27.71 ± 10.53 | 27.63 ± 10.54 | −0.07 | (−0.45 to 0.31) | |
| Minimum Cap | 27 | 31 | 64.88 ± 19.89 | 87.88 ± 38.08 | 24.41 | (6.84 to 41.98) | 0.008 |
| Thickness, μm | | | | | | | |
| Lipid arc, mean | 31 | 35 | 55.94 ± 31.04 | 43.46 ± 3.48 | −12.49 | (−22.17 to −2.80) | 0.013 |
| over frames, ° | | | | | | | |
| % frames with | 83 | 153 | 22.87 ± 29.94 | 23.55 ± 9.94 | 0.66 | (−1.23 to 2.54 | 0.49 |
| normal vessel | | | | | | | |
| % frames with | 83 | 153 | 46.84 ± 29.71 | 43.52 ± 27.75 | −3.28 | (−5.88 to −0.68) | 0.014 |
| fibrous plaque | | | | | | | |
| % frames with | 83 | 153 | 22.14 ± 25.82 | 25.44 ± 27.39 | 3.44 | (1.67 to 5.21) | <0.001 |
| FCa plaque | | | | | | | |
| AutoOCT | | | | | | | |
| ROI Length, mm | 83 | 153 | 27.66 ± 10.55 | 27.59 ± 10.57 | −0.07 | (−0.44 to 0.30) | |
| Minimum Cap | 27 | 31 | 62.86 ± 28.35 | 81.80 ± 33.41 | 18.93 | (15.52 to 22.34) | <0.001 |
| Thickness, μm | | | | | | | |
| Lipid arc, mean | 31 | 35 | 63.12 ± 21.73 | 49.79 ± 20.30 | −13.30 | (15.17 to −11.51) | <0.001 |
| over frames, ° | | | | | | | |
| % frames with | 83 | 153 | 9.68 ± 20.31 | 8.52 ± 18.49 | −1.15 | (−2.52 to 0.22) | 0.099 |
| normal vessel | | | | | | | |
| % frames with | 83 | 153 | 42.16 ± 21.72 | 46.30 ± 22.89 | 4.14 | (1.35 to 6.94) | 0.004 |
| fibrous plaque | | | | | | | |
| % frames with | 83 | 153 | 5.14 ± 5.70 | 6.35 ± 6.07 | 1.21 | (0.40 to 2.02) | 0.004 |
| FCa plaque | | | | | | | |

FCa indicates fibrocalcific plaque; TCFA, thin-cap fibroatheroma; and ThCFA, thick-cap fibroatheroma. (data presented are mean ± SD, percentage [%] as appropriate).

In lesion-level analyses, 39/46 (84.8%) the present system TCFAs at baseline regressed to non-TCFA morphology, compared to 69.2% determined by the core laboratory, whereas only 0.2% of the present system non-TCFA lesions progressed to TCFAs, compared to 1.1% by core laboratory. Consistent with vessel-level findings, the present system mean minimum FCT within each lesion increased from 76.7±36.1 μm to 83.0±35.3 μm compared to 74.0±32.3 μm to 94.2±39.9 μm by the core laboratory. Overall increase in minimum FCT was driven by changes in lesions with TCFA morphology at baseline, with any increase in minimum FCT observed in 82.0% TCFA (92.3% by core laboratory) compared with 58.3% of ThCFA lesions at baseline (52.2% by core laboratory). These results suggest high accuracy of the present system to identify features of drug efficacy, suggesting that the major effect of HIS treatment is to increase the minimum FCT in TCFAs.

The CLIMA (Relationship Between Coronary Plaque Morphology of Left Anterior Descending Artery and Long-Term Clinical Outcome) study undertook OCT imaging of untreated proximal left anterior descending artery arteries with 1 year data available for MACE (composite endpoint of cardiac death and target segment myocardial infarction), with mean lumen area (MLA)<3.5 mm², FCT <75 μm, and lipid arc >180° associated with MACE. We studied 62 participants, comprising 31 MACE and 31 control cases. The present system showed more MACE patients had MLA <3.5 mm² (38.7% vs. 19.5%, p=<0.001), FCT <75 μm (29.0% vs. 12.9%, p=<0.001), and maximum lipid arc >180° (54.8% vs. 41.9%, p=<0.001), similar to core laboratory analysis of our subset (Table 4).

TABLE 4
Present System can detect features of plaque vulnerability

| | | | |
|---|---|---|---|
| All | Patients with | Patients without | |
| Population | Clinical Events | Clinical Events | |
| (n = 62) | (n = 31) | (n = 31) | P-value |

Core Lab OCT findings

| | | | | |
|---|---|---|---|---|
| Minimum lumen area <3.5 | 19 (30.6) | 12 (38.7) | 7 (22.6) | <0.001 |
| mm² (%) | | | | |
| Fibrous cap thickness <75 μm | 18 (29.0) | 13 (41.9) | 5 (16.1) | 0.004 |
| (%) | | | | |
| Maximum lipid arc >180° (%) | 26 (41.9) | 15 (48.4) | 11 (35.5) | <0.001 |

AutoOCT OCT findings

| | | | | |
|---|---|---|---|---|
| Minimum lumen area <3.5 | 18 (29.0) | 12 (38.7) | 6 (19.4) | <0.001 |
| mm² (%) | | | | |
| Fibrous cap thickness <75 μm | 13 (21.0) | 9 (29.0) | 4 (12.9) | <0.001 |
| (%) | | | | |
| Maximum lipid arc >180° (%) | 30 (48.4) | 17 (54.8) | 13 (41.9) | <0.001 |

Clinical events defined as a composite of cardiac death and target vessel myocardial infarction.
p-values given for reference.

Although the sensitivity and specificity of each OCT criteria varied, the positive and negative predictive value and diagnostic accuracy of each variable measured by the present system and the core laboratory were similar (Table 5), suggesting that the present system can identify features of plaque vulnerability similar to a core laboratory.

TABLE 5
Accuracy of Present System to detect higher-risk plaque
features compared to expert core laboratory

| | Sensitivity (%) | Specificity (%) | PPV (%) | NPV (%) |
|---|---|---|---|---|
| Core Laboratory | | | | |
| Minimum lumen area <3.5 mm² | 27.7 | 86.0 | 6.8 | 96.9 |
| Minimum fibrous cap | 40.6 | 83.9 | 8.6 | 97.4 |
| thickness <75 μm | | | | |
| Maximum lipid arc extension >180° | 46.9 | 65.6 | 4.8 | 97.1 |
| AutoOCT | | | | |
| Minimum lumen area <3.5 mm² | 36.7 | 80.0 | 6.5 | 97.1 |
| Minimum fibrous cap | 30.0 | 86.7 | 7.9 | 97.0 |
| thickness <75 μm | | | | |
| Maximum lipid arc extension >180° | 56.7 | 56.7 | 4.6 | 97.2 |

NPV, negative predictive value; PPV, positive predictive value

In summary, a deep learning AI-based image analysis system was designed and tested for intracoronary OCT, validated both internally and externally to detect and measure multiple markers of disease progression/regression and high-risk plaques, and not be limited by poor image quality or imaging artifacts. Importantly, the present system was trained using whole pullbacks representative of real-world clinical practice, and not just perfect, artifact-free images with classical architecture features and known measurements. We find that (a) the present system could recover images containing common artifacts, and the present system-derived plaque classification correlated well with histology; (b) the present system-derived identification and measurement of higher risk features such as FCT and lipid arc were comparable to histopathology, correlated well with an expert reader, and accurately identified TCFA (83.6%); (c) the present system replicated core laboratory findings consistent with plaque stabilization after high-intensity statin use; (d) the present system replicated core laboratory findings of plaque vulnerability, namely MLA <3.5 mm², FCT <75 μm, and lipid arc >180°; (e) Artifact-corrected, segmented images and measurements were available in under 2 minutes/pullback.

Despite the success of deep learning models, many models are trained and tested with small curated datasets with limited diversity, with highly selected frames that include common artifacts due to stents, poor image quality, thrombus, plaque rupture, dissection, haematoma, and bifurcations which may not represent real-world algorithm performance. In contrast, the present system was trained with whole unselected pullbacks (average 285 frames/patient), which is crucial for generalizability and real-world application, and used pre-processing to mitigate effects of artifacts, optimize poor quality images, and allow analysis of all available data. The present system has a number of other features that may improve performance. Many studies only report identification of individual plaque components or artery lumen and not plaque phenotype, which requires simultaneous identification of multiple features. The present system uses a multiclass segmentation design followed by measurement tools based on calibrated images, which allows multiple tissue types to be identified with measurements allowing plaque classification (e.g. AIT vs. PIT, TCFA vs ThCFA). Finally, many studies lack external validation against histopathology, and almost all lack validation against core laboratory analyses of individual frames. In contrast, we used a well-curated database of real-world clinical OCT pullbacks from three centers for training, a separate database for internal validation, and externally validated the present system against two large-scale landmark clinical trials. While there is still room for improvement, the current algorithm replicated core laboratory performance in external validation.

Our study demonstrates that AI-based OCT analysis may aid drug and device development, and trial design and analysis for natural history studies. For example, increased FCT, reduced lipid arc, TCFA regression, and reduced ThCFA progression can be a ‘signature’ of a drug or device likely to reduce MACE. Use of AI-based analyses requires accurate and reproducible analysis of these features, preferably with co-registration of baseline and follow-up images, with vessel and frame-based analysis allowing definitive positive or negative results from small numbers of patients over a relatively short time-frame. The present system measurements showed high accuracy, identifying features of drug efficacy and changes in plaque morphology in response to high-intensity statins in 83 patients treated for 13 m, with changes comparable to the expert core laboratory.

Histopathology and multiple imaging studies have identified the substrate underlying many MACE. However, clinical natural history studies require many hundreds to thousands of patients, often studied for 3-5 years before precursor features of MACE are identified. Analysis of these studies is laborious, time-consuming, and requires expert interpretation. Our model was trained with 36,212 frames from 127 non-selected clinical pullbacks and was able to utilize clinical measurements to classify ex-vivo pullbacks with a frame-level accuracy to detect TCFA of 83.6%. Although OCT provides incremental prognostic information with a high negative predictive value, studies show a high prevalence of vulnerable plaque features but a low positive predictive value. We report a PPV for detecting TCFA ex-vivo of 53.4%, representing enhanced performance compared to an expert-reader. We also show non-inferior diagnostic performance to detect vulnerable plaque features in our automated analysis of CLIMA. While AI-based CT analysis may not replace core laboratories, whole vessel and frame-based analysis in minutes/pullback may greatly speed up the analysis process.

Clinical imaging analysis algorithms are increasingly being used to guide patient management, and in the future may include treatment of high-risk but non-stenotic plaques. However, while prophylactic stenting of higher-risk non-culprit lesions might reduce MACE, their identification in real time is challenging, time-consuming, and human factors including expertise affect measurement accuracy. While AI-based fast, reproducible identification of higher-risk lesions can aid clinical decisions, the software will need to be integrated into current imaging systems, and co-registered with both the OCT and angiography.

We have developed and validated a highly generalizable deep learning artificial intelligence-led model utilizing real-world clinical data as a framework for automatic plaque characterization in coronary OCT. Our model has sufficient sensitivity to demonstrate the small changes in plaque composition seen with pharmacotherapy as well as identify clinical features of plaque vulnerability. Our model may reduce subjectivity in image interpretation through the use of artificial intelligence and image pre-processing to optimize poor quality OCT images containing artifacts, and facilitate real-time quantification of plaque composition with potential applications in research and the management of coronary disease.

At least some of the example embodiments described herein may be constructed, partially or wholly, using dedicated special-purpose hardware. Terms such as ‘component’, ‘module’ or ‘unit’ used herein may include, but are not limited to, a hardware device, such as circuitry in the form of discrete or integrated components, a Field Programmable Gate Array (FPGA) or Application Specific Integrated Circuit (ASIC), which performs certain tasks or provides the associated functionality. In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. Although the example embodiments have been described with reference to the components, modules and units discussed herein, such functional elements may be combined into fewer elements or separated into additional elements. Various combinations of optional features have been described herein, and it will be appreciated that described features may be combined in any suitable combination. In particular, the features of any one example embodiment may be combined with features of any other embodiment, as appropriate, except where such combinations are mutually exclusive. Throughout this specification, the term “comprising” or “comprises” means including the component(s) specified but not to the exclusion of the presence of others.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. An automated computer-implemented method for analyzing a set of images of a coronary artery tissue, the method comprising:
for each image in the set of images, segmenting the image for artery features, including one or more of lumen, side branches and external elastic lamina (EEL), and OCT appearances, including one or more of stent, guide catheter and guidewire shadow, using a first neural network;
when the image is segmented, correcting artifacts in the images by implementing an artifact correction methodology, using a second neural network;
for the artifact corrected image, segmenting the diseased tissue into distinct tissue types using a third neural network, wherein the distinct tissue types include at least one of fibrous tissue, lipid-rich tissue, and calcific tissue;
identifying and measuring features of interests of the segmented tissue types, wherein the features of interests include one or more of arc, thickness, area, and depth for each tissue type;
compiling a first set of measurements for each identified feature of interest from a first subset of images captured at a first time, and a second set of measurements for the same feature of interest from a second subset of images captured at a second time subsequent to the first time; and
determining, using the compiled first and second sets of measurements, changes in the coronary artery tissue, wherein the changes are indicative of progression or regression of a diseased state, to be utilized for determining an efficacy of drug or device therapy, or, using a single set of images, prediction of multiple adverse cardiovascular events (MACE) such as cardiac death or myocardial infarction to help guide treatment.

2. The method as claimed in claim 1, wherein the artifact correction methodology further comprises optimizing image quality using an optimization procedure, the optimization procedure comprising:
converting the set of images to greyscale;
performing lumen masking on the greyscale images;
applying a polar transform to the lumen-masked images from a central lumen point to generate a plurality of panels;
measuring the mean pixel intensity of each panel;
conducting histogram matching of the panels in 2D and 3D based on the brightest panel to align pixel intensity distributions;
integrating the histogram-matched panels into a single image;
reconstructing the image using a cartesian transform along with the lumen mask; and
generating an output image where the images are normalized and corrected for artifacts.

3. The method as claimed in claim 2, wherein the artifact correction methodology further comprises enhancing image clarity by applying a median filter to the integrated image.

4. The method as claimed in claim 2, wherein the artifact correction methodology further comprises using adaptive filtering with binary masks derived from thresholding operations for processing the set of images.

5. The method as claimed in claim 1, wherein the second neural network is configured with machine learning algorithms, including supervised classification models.

6. The method as claimed in claim 1, wherein the third neural network is configured to implement a convolutional neural network (CNN) architecture for extracting and hierarchically organizing features from the set of images to segment the coronary artery tissue into the distinct tissue types.

7. The method as claimed in claim 1, further comprising implementing a combination of spatial filtering, intensity normalization, and edge detection techniques to facilitate tissue segmentation.

8. The method as claimed in claim 1, further comprising applying a fourth neural network to analyze the compiled first and second sets of measurements for determining the efficacy of drug or device therapy, wherein the fourth neural network utilizes a predictive model trained on historical data correlating tissue characteristics with patient outcomes, including myocardial infarction and cardiac death.

9. The method as claimed in claim 1, wherein measuring the features of interest of the segmented tissue types comprises quantifying morphological and textural properties of each tissue type, including one or more of fibrous cap integrity, lipid pool heterogeneity, and calcification patterns, to provide an assessment of plaque composition.

10. The method as claimed in claim 1, wherein the set of images comprises optical coherence tomography (OCT) images of the coronary artery tissue, the OCT images being pre-processed to normalize lighting conditions and contrast levels before analysis.

11. The method as claimed in claim 1, wherein the first and second subsets of images are aligned and co-registered using anatomical landmarks within the coronary artery tissue.

12. The method as claimed in claim 1 wherein each image is of the coronary artery tissue of a patient, and wherein the method comprises determining, using the measurement of each identified feature of interest, a likelihood of the patient having a particular manifestation of a coronary artery disease.

13. The method as claimed in claim 11, wherein the coronary artery disease manifestations include myocardial infarction and cardiac death, and the identified feature of interest is fibrous tissue thickness or lipid.

14. A non-transitory data carrier carrying code which, when implemented on a processor, causes the processor to perform the method of claim 1.

15. An apparatus for analyzing a set of images of a coronary artery tissue, the apparatus comprising:
an imaging device for capturing a set of images of a coronary artery; and
a memory configured to store the set of images;
at least one processor, coupled to memory, arranged to perform steps of the method of claim 1.
